(19) United States
(12) Patent Application Publication
(10) Pub. No.: US 2017/0067973 A1
(43) Pub. Date: Mar. 9, 2017

(54) COIL ASSEMBLY AND MAGNETIC RESONANCE IMAGING APPARATUS INCLUDING THE SAME

(71) Applicant: Samsung Electronics Co., Ltd, Gyeonggi-do (KR)

(72) Inventors: Eun Je Hyun, Gyeonggi-do (KR); Seul Gi Park, Gyeonggi-do (KR); Ju Hyung Lee, Gyeonggi-do (KR); Won Cheol Jeong, Gyeonggi-do (KR)

(21) Appl. No.: 15/256,549

(22) Filed: Sep. 3, 2016

(30) Foreign Application Priority Data
Sep. 3, 2015 (KR) 10-2015-0124737

(51) Int. Cl. G01R 33/34; A61B 5/055; G01R 33/3415

(52) U.S. Cl. CPC G01R 33/34084; G01R 33/3415; A61B 5/055; A61B 2562/164; A61B 2562/166

(57) ABSTRACT

A coil assembly and a magnetic resonance imaging (MRI) apparatus including the same are disclosed. After completion of inspection or repair of the coil assembly, the coil assembly is easily coupled to the Mill apparatus. The coil assembly is configured to interact with a magnetic field generated from a MM apparatus and includes a first case configured to be bendable, a second case configured to be bendable and detachably coupled to the first case, and a printed circuit board substrate disposed between the first case and the second case.

### CROSS-REFERENCE TO RELATED APPLICATION(S) AND CLAIM OF PRIORITY

The present application is related to and claims the benefit of Korean Patent Application No. 2015-0124737, filed on Sep. 3, 2015 in the Korean Intellectual Property Office, the disclosure of which is incorporated herein by reference.

### TECHNICAL FIELD

Embodiments of the present disclosure relate to a coil assembly and a magnetic resonance imaging (MRI) apparatus including the same.

### BACKGROUND

An image processing apparatus (e.g., a medical imaging device) is a device which acquires information of a patient and provides an image of the acquired information so as to diagnose various diseases. For example, the image processing apparatus includes an ultrasonic diagnostic device, an X-ray tomography device, a magnetic resonance imaging (Mill) device, a medical diagnostic device, and the like. The MM apparatus among the above-mentioned devices provides a relatively free imaging condition, high contrast in soft tissue, and a variety of diagnostic information images. Accordingly, the Mill apparatus occupies a prominent place in the medical image diagnostic field.

The MRI apparatus is an image diagnosis device that supplies a uniform frequency and energy to atomic nuclei in a state in which a uniform magnetic field is applied to the atomic nuclei and converts energy emitted from the atomic nuclei into a signal to diagnose the interior of the human body.

A coil assembly may be located at a specific part of a patient to be imaged. The coil assembly receives signals from the human body and obtains images from a specific part. In this case, the coil assembly should adhere closely to the human body, so that it can obtain high-quality images from the human body.

### SUMMARY

To address the above-discussed deficiencies, it is a primary object to provide a coil assembly capable of being easily repaired, and a MM apparatus including the same. As a result, the Mill apparatus obtains high-quality images using a bendable coil assembly.

Additional aspects of the invention will be set forth in part in the description which follows and, in part, will be obvious from the description, or may be learned by practice of the invention.

In accordance with one aspect of the present disclosure, a coil assembly configured to interact with a magnetic field generated from a Mill apparatus includes a first case configured to be bendable, a second case configured to be bendable, and detachably coupled to the first case, and a printed circuit board (PCB) substrate disposed between the first case and the second case.

The first case and the second case may be detachably coupled to each other by a coupling member.

A locking state of the coupling member may be released when the PCB substrate is repaired or inspected, such that the first case and the second case are separated from each other.

The coupling member may re-enter the locking state so as to couple the first case to the second case.

When the first case and the second case are coupled to each other, the PCB substrate may be located between the first case and the second case.

The coupling member may include a snap-fit connector structure.

The coupling member may include a Velcro fabric hook and loop fastener.

The coupling member may include a zipper.

The PCB substrate may be covered by the first case and the second case.

The first case and the second case may be injection-molded by flexible form.

The PCB substrate may include a power feeding PCB configured to receive and amplify a biological signal.

The PCB substrate may include a shield frame covering the power feeding PCB.

The shield frame may be formed by rigid material.

The PCB substrate may include a circuit pattern arranged thereon, and the power feeding PCB may be arranged on the circuit pattern.

The PCB substrate may be configured to be a flexible PCB.

In accordance with another aspect of the present disclosure, a magnetic resonance imaging apparatus includes: a magnet assembly configured to generate a magnetic field; and a coil assembly configured to acquire a magnetic resonance image by interacting with the magnetic field. The coil assembly includes: a case configured to be bendable; and a PCB substrate contained in the case. At least a part of the case is configured to be opened or closed so that the PCB substrate is configured to be withdrawn from or inserted into the case.

The case may be configured to be opened or closed by a coupling member.

The coupling member may include a snap-fit connector structure.

The coupling member may include a zipper.

The coupling member may include a Velcro fabric hook and loop fastener.

Before undertaking the DETAILED DESCRIPTION below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller” means any device, system or part thereof that controls at least one operation, such a device may be implemented in hardware, firmware or software, or some combination of at least two of the same. It should be noted that the functionality associated with any particular controller may be centralized or distributed, whether locally or remotely. Definitions for certain words and phrases are provided throughout this patent document, those of ordinary skill in the art should understand that in many, if not most instances, such definitions apply to prior, as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 is a view illustrating a MRI apparatus according to an embodiment of the present disclosure.

FIG. 2 is a view illustrating a coil assembly according to an embodiment of the present disclosure.

FIG. 3 is an exploded perspective view illustrating the coil assembly according to an embodiment of the present disclosure.

FIG. 4 is a view illustrating a PCB assembly of the coil assembly according to an embodiment of the present disclosure.

FIG. 5 is a view illustrating some parts of the case of the coil assembly according to an embodiment of the present disclosure.

FIG. 6 is a view illustrating some parts of the case of the coil assembly according to another embodiment of the present disclosure.

FIG. 7 is a view illustrating some parts of the case of the coil assembly according to another embodiment of the present disclosure.

### DETAILED DESCRIPTION

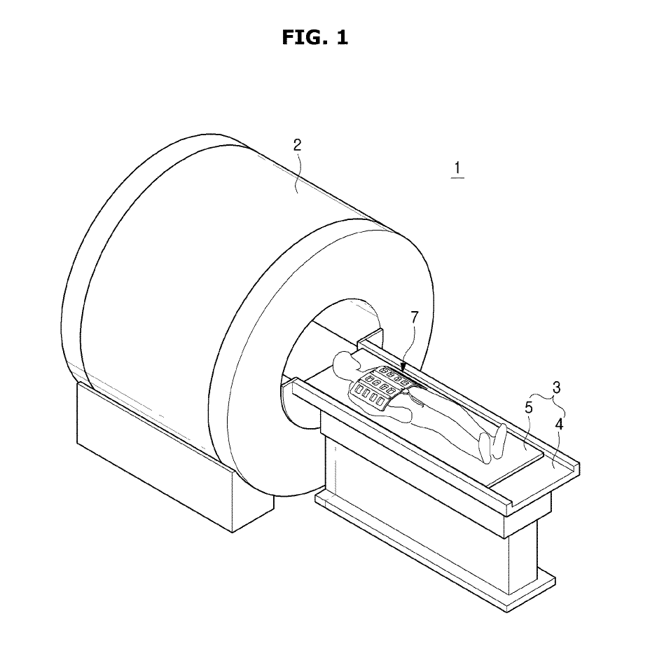

FIGS. 1 through 7, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably arranged imaging system or device. Reference will now be made in detail to the embodiments of the present invention, examples of which are illustrated in the accompanying drawings, wherein like reference numerals refer to like elements throughout. The coil assembly and the MM apparatus including the same according to one embodiment will hereinafter be described with reference to the attached drawings.

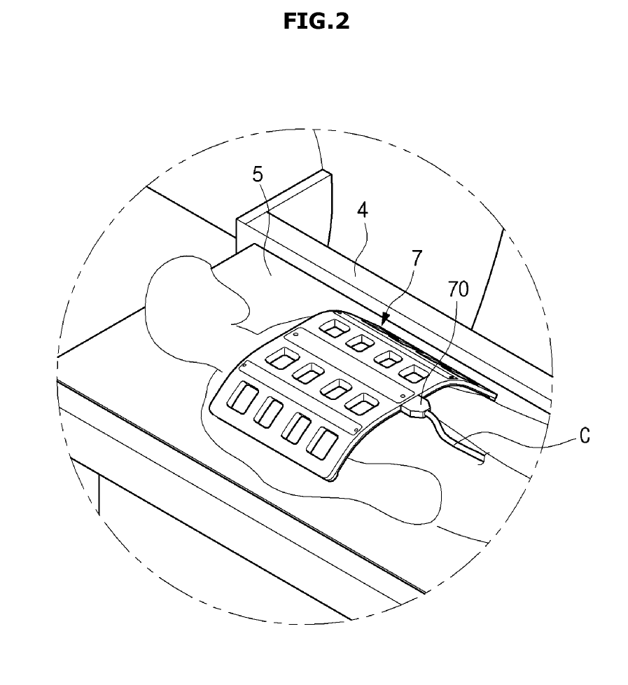

FIG. 1 is a view illustrating a MM apparatus according to an embodiment of the present disclosure. FIG. 2 is a view illustrating a coil assembly according to an embodiment of the present disclosure.

Referring to FIGS. 1 and 2, the MRI apparatus 1 according to one embodiment non-invasively obtains an MRI image including information regarding biological tissues of a patient. Most parts of the human body are formed of water. If strong magnetic field is applied to the patient's body, a time consumed when hydrogen atoms of the patient body absorb specific energy by resonance phenomenon and is then emitted from the hydrogen atom is different between a normal cell and a disease cell. The MRI apparatus 1 may acquire and analyze information regarding the above-mentioned phenomenon, thereby acquiring a magnetic resonance (MR) image regarding a desired part.

The MM apparatus 1 according to an embodiment may include a magnet assembly 2 and a patient table 3. The patient table 3 includes a fixing portion 4 and a transfer portion 5. The transfer portion 5 may be inserted into the magnet assembly 2. The transfer portion 5 on which the patient lies down may move forward or backward. The transfer portion 5 may move toward the magnet assembly 2 so that a desired part of the patient can be imaged.

The MM apparatus 1 may acquire the magnetic resonance (MR) image signal from a target part of the patient through interaction among a main magnetic field caused by a main magnet of the magnet assembly 2, a gradient magnetic field caused by a gradient coil, and the coil assembly 7 attached to the body of the patient who lies on the patient table 3, and may then transmit the MR image signal to the control unit.

The coil assembly 7 may function as a detector configured to receive a biological signal from the patient. The coil assembly 7 may adhere closely to a target part from which a magnetic resonance image will be obtained. For example, if a breast image of the patient is needed, the coil assembly 7 may adhere closely to the breast part of the patient. If images regarding arms or legs of the patient are needed, the coil assembly 7 may be located at the arm or leg of the patient.

The coil assembly 7 may be configured to be bendable by external force. The coil assembly 7 may be bendable to adhere closely to a target object of the patient.

A terminal portion 70 connected to a cable C may be mounted to one side of the coil assembly 7. The coil assembly 7 and the control unit may be interconnected through the cable C. Operations of the coil assembly 7 can be controlled by the control unit.

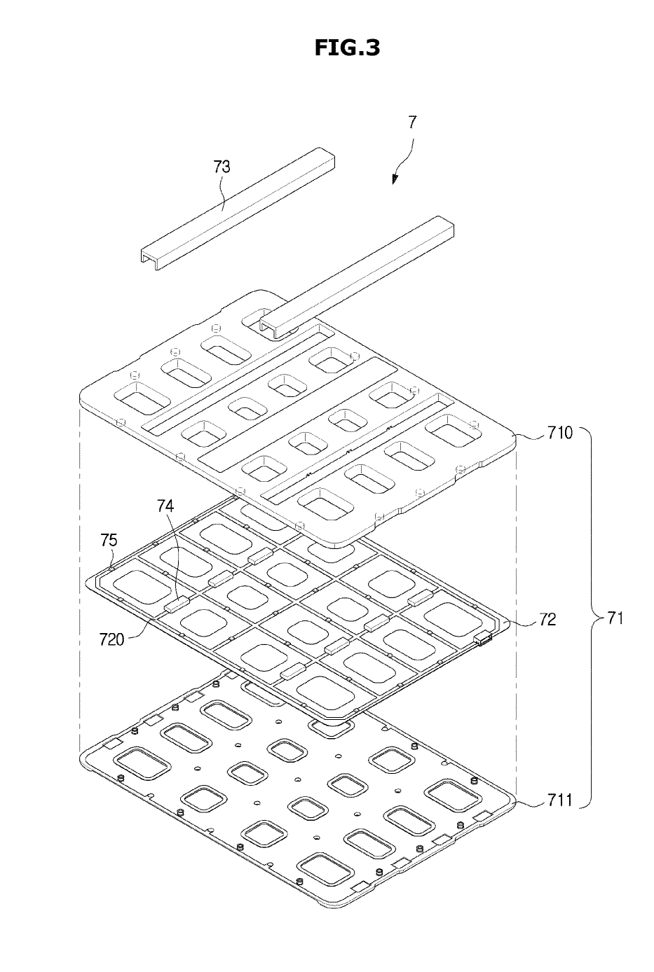

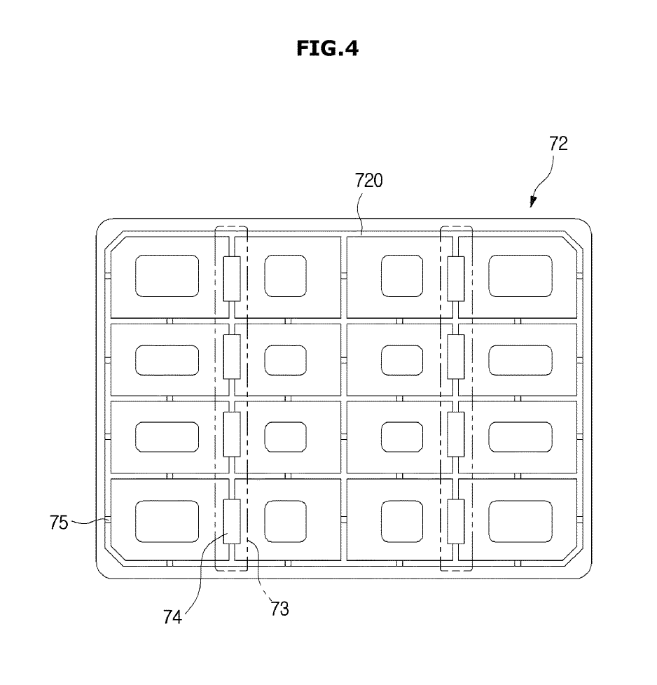

FIG. 3 is an exploded perspective view illustrating the coil assembly according to an embodiment of the present disclosure. FIG. 4 is a view illustrating a PCB assembly of the coil assembly according to an embodiment of the present disclosure.

Referring to FIGS. 3 and 4, the coil assembly 7 according to an embodiment may include a case 71 and a PCB substrate 72. The PCB substrate 72 may be contained in the case 71.

The PCB substrate 72 may be configured in the form of a flexible PCB. The PCB substrate 72 is configured to be bendable so that the PCB substrate 72 can be bent along with the case 71.

A circuit pattern 720 may be printed on the PCB substrate 72. A power feeding PCB 74, a detune PCB 75, etc. may be arranged on the circuit pattern 720. The feeding PCB 74 may amplify biological signals generated from the human body. The detune PCB 75 may cut off connection of the coil when the target part of the patient is not imaged.

The PCB substrate 72 may include a plurality of electronic components therein. For example, at least one detune PCB 75 may be contained in the PCB substrate 72. The detune PCBs 75 may be spaced apart from each other by a predetermined distance over the entire circuit pattern 720.

At least one feeding PCB 74 may be contained in the PCB substrate 72. The feeding PCBs 74 may be spaced apart from each other by a predetermined distance over the circuit pattern 720.

The feeding PCB 74 may be protected by a shield frame 73. The shield frame may be formed by rigid material, such that the shield frame can prevent the feeding PCB from being damaged or broken when the coil assembly 7 is bent. One shield frame 73 may cover the plurality of feeding PCBs 74, or may cover only one feeding PCB 74.

The case 71 may include a first case 710 to cover one surface of the PCB substrate 72 and a second case 711 to cover the other surface of the PCB substrate 72. The first case 710 and the second case 711 may be formed by injection-molding flexible foam such as polyurethane or the like. The first case 710 and the second case 711 may be configured to be bendable.

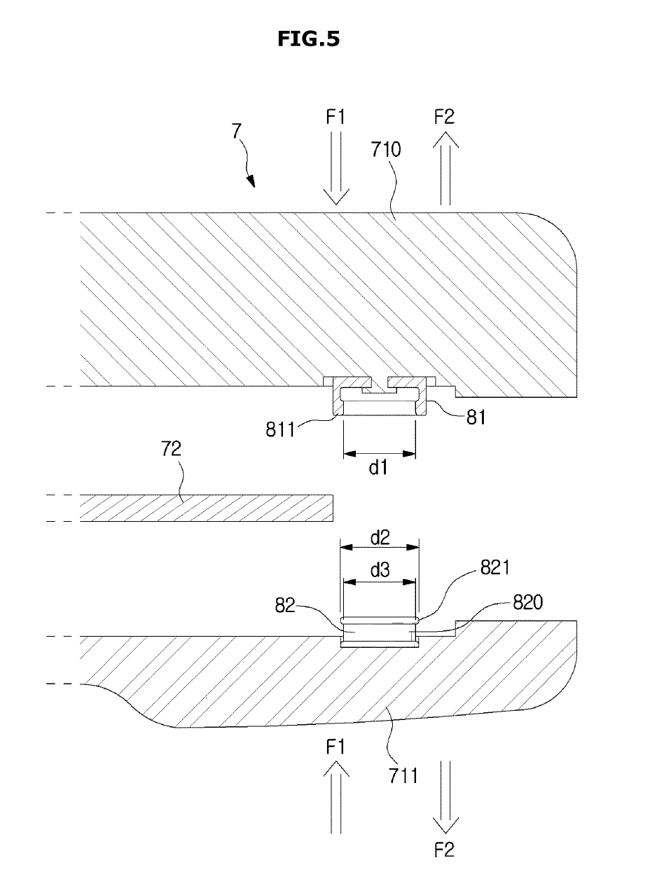

FIG. 5 is a view illustrating some parts of the case of the coil assembly according to an embodiment of the present disclosure.

Referring to FIG. 5, the first case 710 may be coupled to the second case 711 through the coupling portion 8. The PCB substrate 72 may be located between the first case 710 and the second case 711. The first case 710 and the second case 711 may be detachably coupled to each other.

The coupling portion 8 for detachably coupling the first case 710 and the second case 711 may be formed in various shapes. For example, the first case 710 may be coupled to the second cast 71 through a snap-fit structure. For example, a protruded extension portion 81 may be disposed at one surface of the first case 710, and a projection portion 82 capable of interfering with the extension portion 81 may be disposed at one surface of the second case 711.

The extension portion 81 may have elasticity by which the extension portion 81 is bendable outward by external force. The extension portion 81 may include an accommodation portion 810 in which some parts of the projection portion 82 can be accommodated. An interference portion 811 curved inward may be mounted to the end of the extension portion. The interference portion 811 may interfere with the projection portion 82.

The projection portion 82 may include a first projection portion 820 protruding from the second case 711 and a second projection portion 821 mounted to the end of the first projection portion 820. A diameter (d3) of the second projection portion 821 may be smaller than a diameter (d3) of the first projection portion 820. A diameter (d1) of the interference portion 811 may be smaller than a diameter (d3) of the first projection portion 821. A diameter (d3) of the second projection 821 may be identical to or less than a diameter (d1) of the interference portion 811.

In order to couple the first case 710 to the second case 711, the first case 710 and the second case 711 are located in a manner that the extension portion 81 and the projection portion 82 face each other, and the PCB substrate 72 may be located between the first case 710 and the second case 711. If external force (F1) is applied to the external surface of the first case 710 and the external surface of the second case 711, the interference portion 811 slides along the external surface of the first projection portion 820, and at the same time can be modified in structure in a manner that the diameter (d1) of the interference portion 811 increases in size. If the interference portion 811 passes through the first projection portion 820, the interference portion 811 may return to an original state thereof in a manner that the interference portion 811 has the initial diameter (d1) before modification. The diameter (d1) of the interference portion 811 is smaller than the diameter (d2) of the first projection portion 821, such that the interference portion 811 may interfere with the second projection portion 821. As a result, the first case 710 can be coupled to the second case 711.

The first case 710 and the second case 711 may be separated from each other by force (F2) opposite to external force generated when the first case 710 or the second case 711 is coupled. After the interference portion 811 slides along the external surface of the second projection portion 821 by external force, the interference portion 811 passes through the second projection portion 821 such that the interference portion 811 can be separated from the second projection portion 821. As described above, the first case 710 and the second case 711 can be easily separated from each other.

The first case 710 and the second case 711 are detachably coupled to each other. If a faulty operation or malfunction occurs in the PCB substrate 72, the first case 710 is separated from the second case 711 so that the PCB substrate 72 can be inspected and repaired.

In case of the conventional coil assembly, the case formed to cover the PCB substrate may be injection-molded or junctioned as a unit, resulting in formation of the conventional coil assembly. The coil assembly may be properly bendable according to a photographed part of the patient. Electronic components located at the PCB substrate may be damaged or broken by the repeated bending operation. However, the case covering the substrate according to the conventional art is not detachably coupled. If a malfunction or faulty operation occurs in the PCB substrate, the user may repair the PCB substrate by disjointing the case, and must re-perform injection molding of the case so as to cover the entire PCB substrate.

In accordance with the present disclosure, the first case 710 and the second case 711 covering the PCB substrate 72 may be detachably coupled to each other by the coupling structure. Therefore, assuming that malfunction or faulty operation occurs in the PCB substrate 72, the PCB substrate 72 is repaired by separating the first case 710 from the second case 711, and the first case 710 and the second case 711 may then be re-coupled. As described above, the first case 710 and the second case 711 can be easily isolated and coupled to each other, such that the PCB substrate 72 can be easily repaired and maintained. In addition, it is not necessary for the user to perform injection molding of the case 71 after completion of repair of the PCB substrate 72, resulting in reduction of repair charges.

A security member (not shown) may be located at the interconnection of the first case 710 and the second case 711. In order to safely use the coil assembly 7, the security member for indicating whether or not the coil assembly 7 has been opened may be used. As a result, the PCB substrate 72 contained in the case 70 can be prevented from being damaged or broken when the first case 710 and the second case 711 are arbitrarily opened. If electronic components contained in the cases (710, 711) are damaged, a safety problem may occur or normal imaging of a target part of the patient may be difficult. As a result, the user first confirms the operation state of the security member prior to photographing the target part of the patient, and recognizes whether or not the cases (710, 711) have been arbitrarily opened prior to photographing the target part of the patient. Thereafter, the user can acquire images of the target part of the patient.

The security member may be a security sticker mounted to an interconnection part of the first case 710 and the second case 711. The security member may also be implemented as a locking device configured to prevent the first case 710 and the second case 711 from being arbitrarily opened. Categories of the security member are not limited thereto.

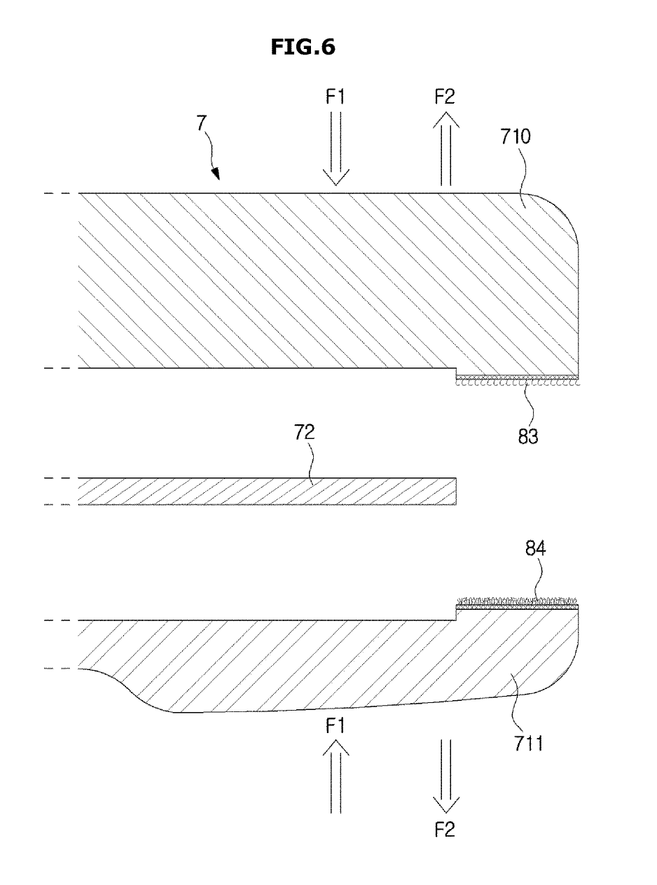

FIG. 6 is a view illustrating some parts of the case of the coil assembly according to another embodiment of the present disclosure.

Referring to FIG. 6, the first case 710 and the second case 711 contained in the coil assembly 7 according to another embodiment may be detachably coupled to each other through a hook and loop fastener, such as, for example, a Velcro fabric hook and loop fastener. A hook portion 83 may be contained in the first case 710, and a loop portion 84 may be contained in the second case 711. In case of the hook portion 83 and the loop portion 84, a crook contained in the hook portion 83 may be caught in a fixing ring contained in the loop portion 84, such that the hook portion 83 and the loop portion 84 may be coupled to each other.

Similar to the embodiment of FIG. 5, the first case 710 is coupled to the second case 711 by external force (F1) generated in the direction along which the first case 710 approaches the second case 711, and the first case 710 may be separated from the second case 711 by the other external force (F2) opposite to the external force (F1).

As described above, since the cases (710, 711) can be detachably coupled to each other, the user can easily and quickly cope with the faulty operation in the PCB substrate 72.

In order to safely use the coil assembly 7, the security member configured to prevent the first case 710 and the second case 711 shown in FIG. 6 from being arbitrarily opened may be used in the same manner as in the embodiment of FIG. 5.

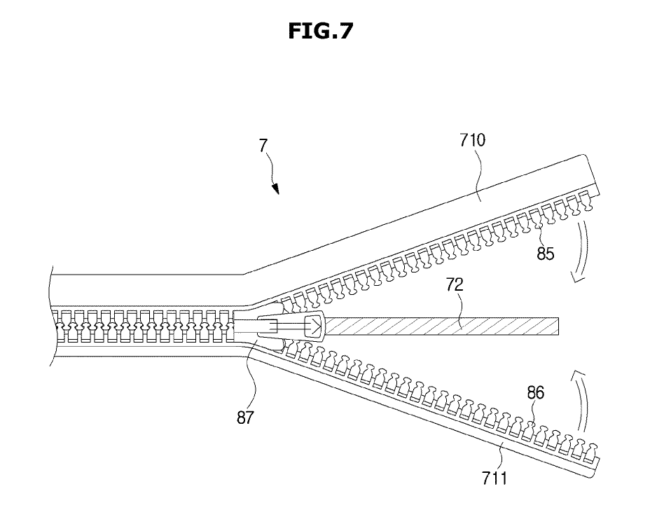

FIG. 7 is a view illustrating some parts of the case of the coil assembly according to another embodiment of the present disclosure.

Referring to FIG. 7, the first case 710 and the second case 711 of the coil assembly 7 may be detachably coupled to each other through zippers (85, 86). Zipper teeth (85, 86) meshed with each other may be respectively mounted to the first case 710 and the second case 711. The PCB substrate 72 may be disposed between the first case 710 and the second case 711. If repair or inspection of the PCB substrate 72 is needed, the user moves a knob 87 contained in the zippers (85, 86) toward an extension direction of the zipper teeth (85, 86), such that the first case 710 and the second case 711 can be separated from each other. If repair or inspection of the PCB substrate 72 is completed, the user moves the knob 87 in the opposite direction to the extension direction, such that the first case 710 may be coupled to the second case 711.

As described above, the first case 710 and the second case 711 may be detachably coupled to each other by zippers. If the first case 710 is coupled to the second case 711 by zippers, saliva secreted from the patient body contacting the coil assembly 7 can be prevented from flowing into the coil assembly 7.

In order to safely use the coil assembly 7, each of the first case 710 and the second case 711 shown in FIG. 7 may include the security member for preventing the first case 710 and the second case 711 from being arbitrarily opened.

The separation and coupling scheme of the case contained in the coil assembly are not limited thereto. At least two of the coupling members shown in FIGS. 5 to 7 may be used as necessary.

The scope or spirit of the coil assembly including the detachably coupled cases is not limited thereto, and the present disclosure can be applied to various coil assemblies. The coil assembly including the detachably coupled cases may be a torso coil for mainly obtaining the breast image of the patient, may be a flex coil for mainly obtaining images regarding arms or legs of the patient, or a loop coil for obtaining images regarding fingers or the like of the patient.

The bendable coil assembly may include a detachably coupled case and a PCB substrate contained in the case. The coil assembly is configured to be bendable so that the coil assembly can adhere closely to the patient body, thereby obtaining a more precise image. The case is detachably coupled, such that the user can easily and quickly cope with the faulty operation or malfunction of the PCB substrate. In addition, the case can be easily coupled by the coupling structure after completion of the repair or inspection of the PCB substrate, resulting in reduction of time and cost consumed for re-performing injection molding of the case.

In addition, although the above-mentioned embodiments have disclosed that the first case and the second case are separated from each other and the PCB substrate contained in each case can be inspected and repaired for convenience of description and better understanding of the present disclosure, the first case and the second case are not completely separated from each other, and may also be opened or closed by the coupling member as necessary. One side of the first case or the second case is configured to be opened or closed, the opening/closing device is opened to take the PCB substrate out of the case, the PCB substrate is repaired or inspected, and the repaired or inspected PCB substrate is inserted into the case, such that the opening/closing device can be locked.

As is apparent from the above description, the coil assembly can be easily decomposed. The coil assembly is decomposed into a plurality of divided sections such that the divided sections are easily assembled after completion of internal inspection or repair. High-quality images for a target object can be obtained using the bendable coil assembly.

Although the present disclosure has been described with an exemplary embodiment, various changes and modifications may be suggested to one skilled in the art. It is intended that the present disclosure encompass such changes and modifications as fall within the scope of the appended claims.

What is claimed is:

1. A coil assembly configured to interact with a magnetic field generated from a magnetic resonance imaging (MRI) apparatus, the coil assembly comprising:
a first case configured to be bendable;
a second case configured to be bendable and detachably coupled to the first case; and
a printed circuit board (PCB) substrate disposed between the first case and the second case.

2. The coil assembly according to claim 1, wherein the first case and the second case are detachably coupled to each other by a coupling member.

3. The coil assembly according to claim 2, wherein a locking state of the coupling member is released when the PCB substrate is repaired or inspected, such that the first case and the second case are separated from each other.

4. The coil assembly according to claim 3, wherein the coupling member is configured to re-enter the locking state so as to couple the first case to the second case.

5. The coil assembly according to claim 4, wherein:
when the first case and the second case are coupled to each other, the PCB substrate is located between the first case and the second case.

6. The coil assembly according to claim 2, wherein the coupling member includes a snap-fit structure.

7. The coil assembly according to claim 2, wherein the coupling member includes a hook and loop fastener.

8. The coil assembly according to claim 2, wherein the coupling member includes a zipper.

9. The coil assembly according to claim 1, wherein the PCB substrate is covered by the first case and the second case.

10. The coil assembly according to claim 1, wherein the first case and the second case are injection-molded by flexible form.

11. The coil assembly according to claim 1, wherein the PCB substrate includes a power feeding PCB configured to receive and amplify a biological signal.

12. The coil assembly according to claim 11, wherein the PCB substrate includes a shield frame covering the power feeding PCB.

13. The coil assembly according to claim 12, wherein the shield frame is formed to include a rigid material.

14. The coil assembly according to claim 11, wherein:
the PCB substrate includes a circuit pattern arranged on the PCB substrate; and
the power feeding PCB is arranged on the circuit pattern.

15. The coil assembly according to claim 1, wherein the PCB substrate comprises a flexible PCB.

16. A magnetic resonance imaging apparatus comprising:
a magnet assembly configured to generate a magnetic field; and
a coil assembly configured to acquire a magnetic resonance image by interacting with the magnetic field,
wherein the coil assembly includes:
a case configured to be bendable; and
a printed circuit board (PCB) substrate contained in the case, and
wherein at least a part of the case is configured to be opened or closed for the PCB substrate to be withdrawn from or inserted into the case.

17. The magnetic resonance imaging apparatus according to claim 16, wherein the case is configured to be opened or closed by a coupling member.

18. The magnetic resonance imaging apparatus according to claim 17, wherein the coupling member includes one of a snap-fit structure, a zipper, or a hook and loop fastener.
